(19) United States
(12) Patent Application Publication
(10) Pub. No.: US 2009/0204114 A1
(43) Pub. Date: Aug. 13, 2009

(54) Electrosurgical Forceps with Slow Closure Sealing Plates and Method of Sealing Tissue

(75) Inventor: Darren Odom, Longmont, CO (US)

Correspondence Address: Tyco Healthcare Group LP, 60 MIDDLETOWN AVENUE, NORTH HAVEN, CT, 06473, US

(73) Assignee: Covidien AG

(21) Appl. No.: 12/425,244

(22) Filed: Apr. 16, 2009

Related U.S. Application Data
(63) Continuation-in-part of application No. 12/359,571, filed on Jan. 26, 2009.
Application No. 12/359,571 is a continuation of application No. 11/095,123, filed on Mar. 31, 2005, now Pat. No. 7,491,202.

(51) Int. Cl. A61B 18/14

(52) U.S. Cl. 606/51

(57) ABSTRACT

An electrosurgical system for sealing tissue includes electrosurgical bipolar forceps and an electrosurgical generator. The electrosurgical bipolar forceps includes a shaft member having an end effector assembly disposed at a distal end. The end effector assembly includes jaw members movable from a first position in spaced relation relative to one another to a subsequent position. The jaw members are adapted to cooperate to grasp tissue therebetween. The jaw members each include a sealing plate that communicates electrosurgical energy through the held tissue. The gap sensor is adapted to sense the gap distance between the jaw members. The electrosurgical generator is operatively coupled to the electrosurgical bipolar forceps and generates the electrosurgical energy. The electrosurgical generator is in operative communication with the gap sensor and monitors a sensed gap distance therefrom. The electrosurgical generator generates the electrosurgical energy based on the sensed gap distance as a function of time.

### CROSS-REFERENCE TO RELATED APPLICATIONS

This application claims the benefit of and is a continuation-in-part of U.S. patent application Ser. No. 12/359,571 filed on Jan. 26, 2009 also entitled “ELECTROSURGICAL FORCEPS WITH SLOW CLOSURE SEALING PLATES AND METHOD OF SEALING TISSUE” which is a continuation of U.S. patent application Ser. No. 11/095,123 filed on Mar. 31, 2005 also entitled “ELECTROSURGICAL FORCEPS WITH SLOW CLOSURE SEALING PLATES AND METHOD OF SEALING TISSUE”, now U.S. Pat. No. 7,491,202, both of which are herein incorporated by reference in their entirety.

### BACKGROUND

1. Technical Field

The present disclosure relates to an electrosurgical instrument and method for performing electrosurgical procedures. More particularly, the present disclosure relates to an open or endoscopic bipolar electrosurgical forceps including opposing jaw members which are configured to slowly close about tissue and a method of using the forceps to perform so-called “slow close” tissue sealing procedures, i.e., the sealing plates are designed to close at a specified rate and/or pressure to create a tissue seal of highest integrity.

2. Background of Related Art

A forceps is a pliers-like instrument which relies on mechanical action between its jaws to grasp, clamp and constrict vessels or tissue. So-called “open forceps” are commonly used in open surgical procedures whereas “endoscopic forceps” or “laparoscopic forceps” are, as the name implies, are used for less invasive endoscopic surgical procedures. Electrosurgical forceps (open or endoscopic) utilize mechanical clamping action and electrical energy to effect hemostasis on the clamped tissue. The forceps include electrosurgical sealing plates which apply the electrosurgical energy to the clamped tissue. By controlling the intensity, frequency and duration of the electrosurgical energy applied through the sealing plates to the tissue, the surgeon can coagulate, cauterize and/or seal tissue.

Tissue or vessel sealing is a process of liquefying the collagen, elastin and ground substances in the tissue so that they reform into a fused mass with significantly-reduced demarcation between the opposing tissue structures. Cauterization involves the use of heat to destroy tissue and coagulation is a process of desiccating tissue wherein the tissue cells are ruptured and dried.

Since tissue sealing procedures involve more than simply cauterizing tissue, to create an effective seal the procedures involve precise control of a variety of factors. In order to affect a proper seal in vessels or tissue, it has been determined that two predominant mechanical parameters must be accurately controlled: the pressure applied to the tissue; and the gap distance between the electrodes (i.e., distance between opposing jaw members when closed about tissue).

Numerous electrosurgical instruments have been proposed in the past for various open and endoscopic surgical procedures. However, most of these instruments cauterize or coagulate tissue and are not designed to create an effective or a uniform seal.

In addition, many of the instruments of the past include blade members or shearing members which simply cut tissue in a mechanical and/or electromechanical manner and are relatively ineffective for vessel or tissue sealing purposes. Other instruments generally rely on clamping pressure alone to procure proper sealing thickness and are often not designed to take into account gap tolerances and/or parallelism and flatness requirements which are parameters which, if properly controlled, can assure a consistent and effective tissue seal. Thus, a need exists to develop an electrosurgical instrument which effectively and consistently seals tissue.

### SUMMARY

The present disclosure relates to an electrosurgical instrument and method for performing electrosurgical procedures. More particularly, the present disclosure relates to an open or endoscopic bipolar electrosurgical forceps including opposing jaw members which are configured to slowly close about tissue and a method of using the forceps to perform so-called “slow close” tissue sealing procedures, i.e., the sealing plates are designed to close at a specified rate and/or pressure to create a tissue seal of highest integrity.

In one embodiment of the present disclosure, an electrosurgical system for sealing tissue includes electrosurgical bipolar forceps and an electrosurgical generator. The electrosurgical bipolar forceps includes a shaft member having an end effector assembly disposed at a distal end. The end effector assembly includes jaw members movable from a first position in spaced relation relative to one another to a subsequent position. The jaw members are adapted to cooperate to grasp tissue therebetween. The jaw members each include a sealing plate that communicates electrosurgical energy through the held tissue. The gap sensor is adapted to sense and monitor the gap distance between the jaw members. The gap sensor may be an optical gap sensor. The electrosurgical generator is operatively coupled to the electrosurgical bipolar forceps and generates the electrosurgical energy. The electrosurgical generator is in operative communication with the gap sensor and monitors a sensed gap distance therefrom. The electrosurgical generator generates the electrosurgical energy such that the sensed gap distance as a function of time.

In another embodiment of the present disclosure, the electrosurgical generator monitors the gap sensor to determine an initial gap distance. The electrosurgical generator generates the electrosurgical energy to tissue such that the gap distance decreases between the jaw members by a predetermined faction of the initial gap distance after a predetermined amount of time. The predetermined fraction of the initial gap distance may be about one-half of the initial gap distance and the predetermined amount of time may be greater than about one-half of a second. The electrosurgical generator utilizes the initial gap distance to determine initial parameters of the electrosurgical energy, e.g., an initial frequency, an initial voltage, an initial current, an initial duty cycle, an initial power and an initial input impedance. Additionally or alternatively, the electrosurgical generator utilizes the initial gap distance to determine at least one of tissue type, jaw fill, tissue density, tissue compliance, tissue thickness, tissue hydration, tissue impedance and tissue impedance per unit volume.

In yet another embodiment of the present disclosure, the electrosurgical generator generates the electrosurgical energy such that the sensed gap distance decreases by a predetermined amount as a function of time when the about constant pressure is applied to the grasped tissue by varying one or more parameters of the electrosurgical energy. The parameters of the electrosurgical energy may be one or more a frequency, a voltage, current, a duty cycle, a power and an input impedance. The electrosurgical generator may utilize a feedback control algorithm. For example, the electrosurgical generator can generate the electrosurgical energy such that the sensed gap distance decreases by about a predetermined rate of distance during a substantial temporal portion of generation of the electrosurgical energy. The feedback control algorithm has the gap distance as an input and the electrosurgical energy is an output. The feedback control algorithm may be a P-I-D control algorithm.

In another embodiment of the present disclosure, one (or more) of the sealing plates includes an adjustable stop member. The adjustable stop member is coupled to a controller of the electrosurgical generator. The adjustable stop member separates the sealing plates by a predetermined gap distance and adjusts the adjustable stop member to close the sealing plates in accordance with the function of time.

In yet another embodiment of the present disclosure, a method for sealing tissue includes: providing electrosurgical bipolar forceps; applying pressure to the tissue grasped by the jaw members (e.g., about constant pressure); sensing the gap distance between the jaw members of the electrosurgical bipolar forceps; communicating electrosurgical energy through the tissue grasped by the jaw members; and adjusting the electrosurgical energy such that the sensed gap distance decreases by a predetermined amount as a function of time. The predetermined amount is about one-half of an initial gap distance. The adjusting step can adjust the electrosurgical energy such that the sensed gap distance decreases by the predetermined amount after about one-half of a second. The method may further include sensing an initial gap distance between the jaw members and/or determining one or more of an initial frequency, an initial voltage, an initial current, an initial duty cycle, an initial power and an initial input impedance utilizing the initial gap distance between the jaw members.

### BRIEF DESCRIPTION OF THE DRAWINGS

Various embodiments of the present disclosure are described herein with reference to the drawings wherein:

FIG. 1A is a perspective view of an endoscopic bipolar forceps which is configured to close at a predetermined rate according to the present disclosure;

FIG. 1B is a side, partial internal view of an endoscopic forceps showing a selectively adjustable stop member assembly according to the present disclosure;

FIG. 1C is an enlarged view of the area of detail of FIG. 1B according to the present disclosure;

FIG. 2 is a side, partial internal view of an end effector assembly shown in closed configuration according to the present disclosure;

FIG. 3 is a rear, perspective view of the end effector of FIG. 2 shown with tissue grasped therein according to the present disclosure;

FIG. 4 is an enlarged, perspective view of an electrically conductive sealing plate of the end effector assembly showing a series of selectively adjustable stop members disposed thereon according to the present disclosure;

FIG. 5 shows a flow chart showing a sealing method using the endoscopic bipolar forceps of FIGS. 1A-4 according to the present disclosure;

FIG. 6 shows a graph illustrating the changes occurring to collagen during sealing utilizing the method shown in FIG. 5 according to the present disclosure;

FIG. 7 is a side, partial internal view of an end effector assembly including a slow close spring mechanism shown in closed configuration according to the present disclosure;

FIG. 8 is a perspective view of an open bipolar forceps which is configured to close at a predetermined rate according to the present disclosure;

FIG. 9 shows a graph illustrating an embodiment of the changes occurring to tissue during sealing using the endoscopic bipolar forceps of FIGS. 1A-4 and 7-8 according to the present disclosure; and

FIG. 10 shows a flow chart showing a sealing method using the endoscopic bipolar forceps of FIGS. 1A-4 and 7-8 according to the present disclosure.

### DETAILED DESCRIPTION

Particular embodiments of the present disclosure will be described hereinbelow with reference to the accompanying drawings. In the following description, well-known functions or constructions are not described in detail to avoid obscuring the present disclosure in unnecessary detail.

Electrosurgical forceps which is configured to have sealing plates designed to close at a predetermined rate based on automatically-induced or manually-induced closure is disclosed. A method for controlling or regulating the sealing plates to close at a selected or predetermined closure rate is also discussed and described herein.

In one particular useful embodiment, the electrosurgical forceps includes at least one selectively adjustable (automatic or manual) stop member which controls the distance between the sealing plates.

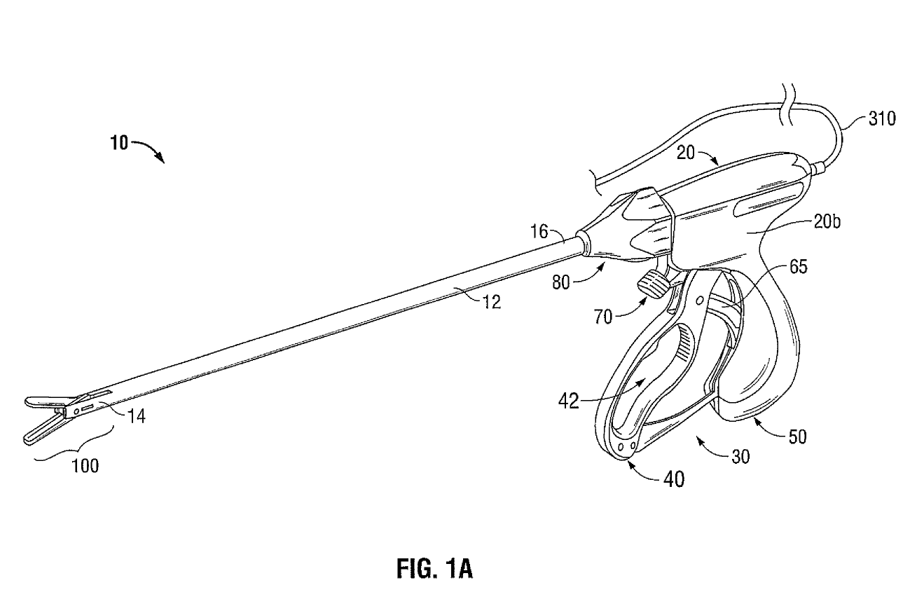

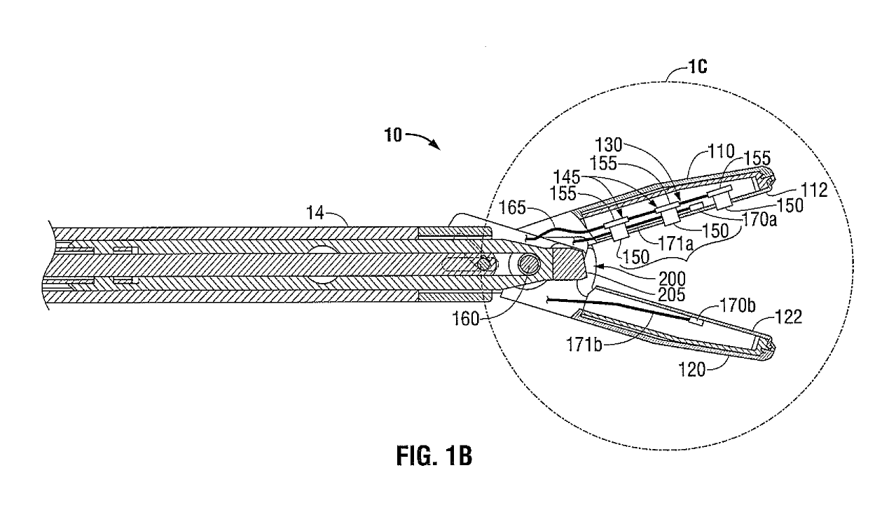

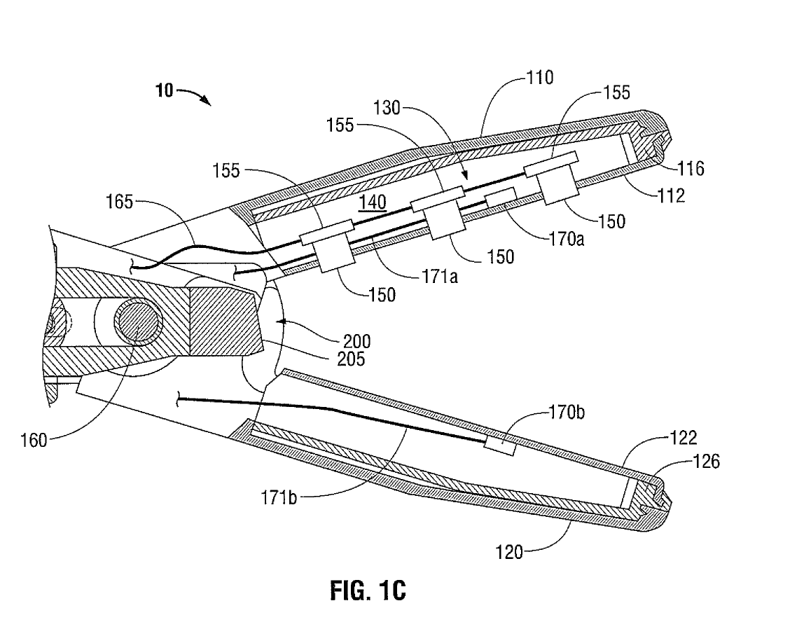

More particularly and with specific reference to the figures, FIG. 1A shows an endoscopic vessel sealing bipolar forceps 10. Those skilled in the art will understand that the invention according to the present disclosure may be adapted for use with either an endoscopic instrument or an open instrument. It should also be appreciated that different electrical and mechanical connections and other considerations apply to each particular type of instrument, however, the novel aspects with respect to the sealing plates configured to close at a predetermined, automatically configured or manually-induced closure rate (hereinafter “slow closure sealing plates”) and their operating characteristics remain generally consistent with respect to both the open or endoscopic designs.

The forceps 10 is shown by way of example and other electrosurgical forceps are also envisioned which allow for slow closure sealing plates of the present disclosure. In the drawings and in the description which follows, the term “proximal”, refers to the end of the forceps 10 which is closer to the user, while the term “distal” refers to the end of the forceps which is further from the user.

FIGS. 1A-1C show the forceps 10 which is configured to support an effector assembly 100. More particularly, forceps 10 generally includes a housing 20, a handle assembly 30, a rotating assembly 80, and a trigger assembly 70 which mutually cooperate with the end effector assembly 100 to grasp, seal and, if required, divide tissue. The forceps 10 also includes a shaft 12 which has a distal end 14 which mechanically engages the end effector assembly 100 and a proximal end 16 which mechanically engages the housing 20 proximate the rotating assembly 80.

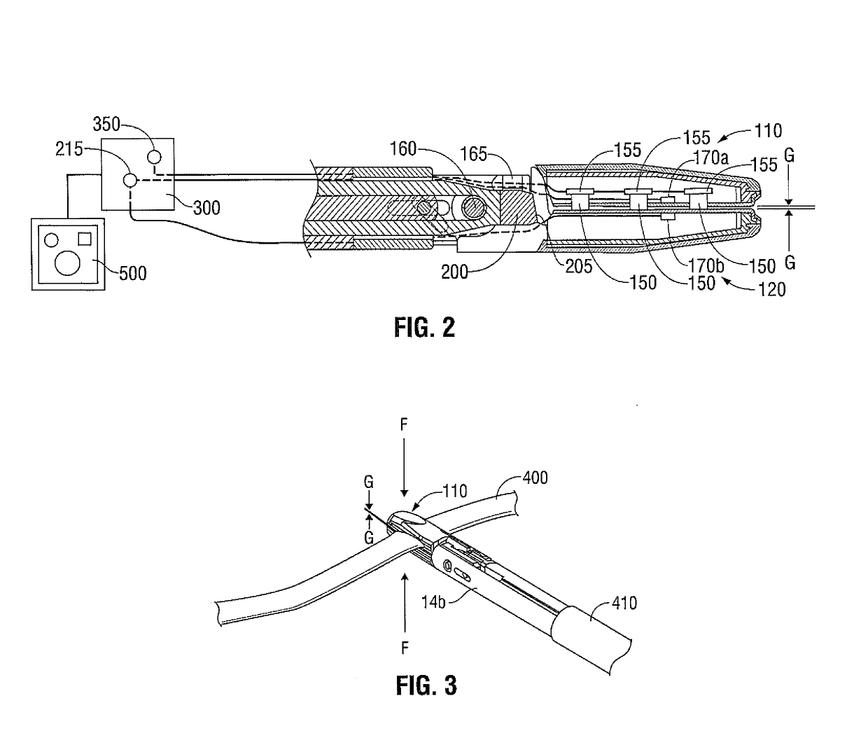

The forceps 10 also includes a plug (not shown) which connects the forceps 10 to a source of electrosurgical energy, e.g., an electrosurgical generator 500, via an electrical cable 310 (See FIG. 2). Handle assembly 30 includes a fixed handle 50 and a movable handle 40. Handle 40 moves relative to the fixed handle 50 to actuate the end effector assembly 100 and enable a user to grasp and manipulate tissue 400 as shown in FIG. 3.

The end effector assembly 100 includes a pair of opposing jaw members 110 and 120 each having an electrically conductive sealing plate 112 and 122, respectively, attached thereto for conducting electrosurgical energy through tissue 400 held therebetween. More particularly, the jaw members 110 and 120 move in response to movement of the handle 40 from an open position to a closed position. In open position the sealing plates 112 and 122 are disposed in spaced relation relative to one another. In a clamping or closed position the sealing plates 112 and 122 cooperate to grasp tissue and apply electrosurgical energy thereto.

The jaw members 110 and 120 are activated using a drive assembly (not shown) enclosed within the housing 20. The drive assembly cooperates with the movable handle 40 to impart movement of the jaw members 110 and 120 from the open position to the clamping or closed position. Examples of a handle assemblies are shown and described in commonly-owned U.S. application Ser. No. 10/389,894 entitled “VESSEL SEALER AND DIVIDER AND METHOD MANUFACTURING SAME” and commonly owned U.S. application Ser. No. 10/460,926 entitled “VESSEL SEALER AND DIVIDER FOR USE WITH SMALL TROCARS AND CANNULAS” which are both hereby incorporated by reference herein in their entirety.

In addition, the handle assembly 30 of this particular disclosure includes a four-bar mechanical linkage which provides a unique mechanical advantage when sealing tissue between the jaw members 110 and 120. For example, once the desired position for the sealing site is determined and the jaw members 110 and 120 are properly positioned, handle 40 may be compressed fully to lock the electrically conductive sealing plates 112 and 122 in a closed position against the tissue. The details relating to the inter-cooperative relationships of the inner-working components of forceps 10 are disclosed in the above-cited commonly-owned U.S. patent application Ser. No. 10/369,894. Another example of an endoscopic handle assembly which discloses an off-axis, lever-like handle assembly, is disclosed in the above-cited U.S. patent application Ser. No. 10/460,926.

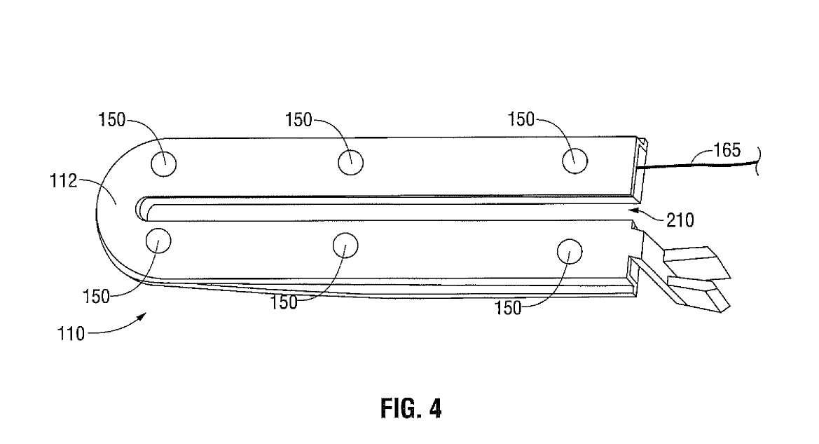

As shown in FIGS. 1A-1C, the forceps 10 also includes a trigger 70 which advances a knife 200 disposed within the end effector assembly 100. Once a tissue seal is formed, the user activates the trigger 70 to separate the tissue 400 along the tissue seal. Knife 200 preferably includes a sharpened edge 205 for severing the tissue 400 held between the jaw members 110 and 120 at the tissue sealing site. FIG. 4 shows a longitudinally-oriented channel 210 defined in an electrically conductive sealing plate 112 extending from the proximal end to the distal end thereof. The channel 210 facilitates longitudinal reciprocation of the knife 200 along a preferred cutting plane to effectively and accurately separate the tissue 400 along a formed tissue seal.

The forceps 10 also includes a rotating assembly 80 mechanically associated with the shaft 12 and the drive assembly (not shown). Movement of the rotating assembly 80 imparts similar rotational movement to the shaft 12 which, in turn, rotates the end effector assembly 100. Various features along with various electrical configurations for the transference of electrosurgical energy through the handle assembly 20 and the rotating assembly 80 are described in more detail in the above-mentioned commonly-owned U.S. patent application Ser. Nos. 10/369,894 and 10/460,926.

As best seen with respect to FIGS. 1A-2, the end effector assembly 100 attaches to the distal end 14 of shaft 12. The jaw members 110 and 120 are preferably pivotable about a pivot 160 from the open to closed positions upon relative reciprocation, i.e., longitudinal movement, of the drive assembly (not shown). Again, mechanical and cooperative relationships with respect to the various moving elements of the end effector assembly 100 are further described by example with respect to the above-mentioned commonly-owned U.S. patent application Ser. Nos. 10/369,894 and 10/460,926.

It is envisioned that the forceps 10 may be designed such that it is fully or partially disposable depending upon a particular purpose or to achieve a particular result. For example, end effector assembly 100 may be selectively and releasably engageable with the distal end 14 of the shaft 12 and/or the proximal end 16 of the shaft 12 may be selectively and releasably engageable with the housing 20 and handle assembly 30. In either of these two instances, the forceps 10 may be either partially disposable or reposable, such as where a new or different end effector assembly 100 or end effector assembly 100 and shaft 12 are used to selectively replace the old end effector assembly 100 as needed.

Since the forceps 10 applies energy through electrodes, each of the jaw members 110 and 120 includes an electrically conductive sealing plate 112 and 122, respectively, disposed on an inner-facing surface thereof. Thus, once the jaw members 110 and 120 are fully compressed about the tissue 400, the forceps 10 is now ready for selective application of electrosurgical energy as shown in FIG. 3. At that point, the electrically conductive plates 112 and 122 cooperate to seal tissue 400 held therebetween upon the application of electrosurgical energy. Jaw members 110 and 120 also include insulators 116 and 126 which together with the outer, non-conductive plates of the jaw members 110 and 120 are configured to limit and/or reduce many of the known undesirable effects related to tissue sealing, e.g., flashover, thermal spread and stray current dissipation as shown in FIG. 1C.

Of particular importance to this disclosure is the slow close system which allows the gap “G” disposed between the sealing plates when the jaw members are disposed in a closed position to close at a predetermined rate. This has been determined to enhance tissue sealing especially when sealing larger tissue structures (e.g., lung, liver, bronchus, bowels, etc.). A slow close activation surgical technique involves activating the surgical instrument and thereafter slowly closing the sealing plates 112 and 122 of the jaw members to grasp and apply pressure to the tissue to affect sealing. As can be appreciated, this type of procedure is very difficult to master manually due to the many variables involved with the sealing process and, as a result, the instrument may short or the sealing cycle may complete prior to obtaining the fully closed ratcheted position. Hence, it is preferred that the procedure is automated using a series of sensors and controllers. It is envisioned that an automatic stop member adjustment system (described below) is one way to achieve slow close activation and provide more effective sealing of large tissue structures. The closure rate may be adjusted during activation based upon a continually-sensed surgical condition (e.g., tissue impedance, tissue type, tissue clarity, tissue compliance, etc.) utilizing a feed back control loop or control source 300, and a sensor assembly 170a and 170b and a mechanically retractable/extendable stop member assembly 140.

With respect to this particular embodiment, it is known that sealing of the tissue 400 is accomplished by virtue of a unique combination of gap control, pressure and electrical control. In other words, controlling the intensity, frequency and duration of the electrosurgical energy applied to the tissue through the sealing plate 112 and 122 are important electrical considerations for sealing tissue. In addition, two mechanical factors play an important role in determining the resulting thickness of the sealed tissue and the effectiveness of the seal, i.e., the pressure applied between the opposing jaw members 110 and 120 (between about 3 kg/cm² to about 16 kg/cm²) and the gap distance “G” between the opposing sealing plates 112 and 122 of the jaw members 110 and 120, respectively, during the sealing process (between about 0.001 inches or higher denoting upon the size of the tissue). A third mechanical factor has recently been discovered which contributes to the quality and consistency of a tissue seal, namely the closure rate of the electrically conductive surfaces or sealing plates during activation.

More particularly, controlling the gap distance “G” between opposing sealing surfaces 112 and 122 directly relates to the closure rate i.e., the closure rate is defined as the rate of change of the gap distance “G.” Therefore, adjusting the gap distance “G” allows the user to adjust the closure rate. As discussed in more detail below, the forceps 10 according to the present disclosure controls the gap distance “G” using one technique which allows a user to selectively adjust (i.e., manually, automatically based on sensed surgical conditions or predetermined parameters) the retraction or extension of at least one stop member 150 relative to the surface of the sealing plate, e.g., 112. As a result thereof adjusting stop member 150 controls the closure rate which, in turn, allows a surgeon to implement a slow close surgical procedure using forceps 10.

More specifically, the rate of closure of the sealing plates 112 and 122 to grasp and/or apply pressure to tissue is regulated by adjusting the gap distance “G” during the surgical procedure. In one particular instance, the stop members 150 are connected to a controller 155 which together comprise a selectively adjustable stop member control unit 145. Each of the stop member control units 145 is connected to the stop member assembly 140 which regulates the gap distance “G” by extending or retracting a plurality of stop members 150 based on the control signals received from the control source 300 and the feedback signals transmitted by a sensor assembly 170a and 170b. The controller 155 electrically, mechanically or electro-mechanically adjusts the distance the stop members 150 project by retracting or extending the stop members 150 from the sealing plate 112. As a result, the gap distance “G” is adjusted by changing the distance that the stop members 150 project from the sealing plate 112. The controller 155 is adapted to receive signals from a control source 300 shown in FIG. 2 which may be attached to an electrosurgical generator 500 or incorporated into the housing of the forceps 10.

As discussed above, the stop member 150 limits the movement of the two opposing jaw members 110 and 120 (and sealing plates 112 and 122) relative to one another by acting as a barrier between the two surfaces. It is envisioned that the stop members 150 may be disposed on one or both of the sealing plates 112 and 122 depending upon a particular purpose or to achieve a particular result. Preferably, the stop members 150 extend from at least one of the sealing plates 112, 122 a predetermined distance according to the specific material properties of the stop member 150 (e.g., compressive strength, thermal expansion, etc.) to yield a consistent and accurate gap distance “G” during sealing.

In order for the stop members 150 to prevent the sealing plates 112, 122 from coming in contact with each other, preferably, the stop members 150 are made from an insulative material, e.g., parylene, nylon and/or ceramic and are dimensioned to limit opposing movement of the sealing plates 112 and 122 to within the above mentioned gap range “G”. However, the compressive strength of the material used in manufacturing the stop member 150 should be considered during activation since one material may have to be adjusted differently from another material to achieve the same gap distance “G”. For example, the compressive strength of nylon is different from ceramic and, therefore, the nylon material may have to extend a greater distance from the sealing plate 112 to counteract the closing force of the opposing jaw members 110 and 120 and to achieve the same desired gap distance “G”. As can be appreciated, these considerations may be automatically regulated or controlled at the control source 300 via a computer algorithm or look up table as discussed in more detail below.

Moreover, it is contemplated that any combination of different stop members 150 may be assembled along the sealing plates 112 (and/or 122) to achieve a desired gap distance “G”. A ceramic or insulative coating may be deposited or sprayed onto the tissue engaging plate of the stop member(s) 150. Thermal spraying techniques are contemplated which involve depositing a broad range of heat-resistant and insulative materials on the tissue engaging plates of the stop members 150, high velocity Oxy-fuel deposition, plasma deposition, etc. Examples of stop members 150, control units 145, and stop member assemblies 140 are shown and described in a commonly-owned U.S. patent application Ser. No. 10/846,262 entitled “TISSUE SEALER WITH NON-CONDUCTIVE VARIABLE STOP MEMBERS AND METHOD OF SEALING TISSUE” which is hereby incorporated by reference herein in its entirety.

FIG. 4 shows one exemplary configuration of the stop members 150 disposed on or protruding from the sealing plate 112. It is envisioned that the stop members 150 can be positioned on either or both jaw members 110 and 120 depending upon a particular purpose or to achieve a desired result. More particularly and as illustrated in FIG. 4, a series of longitudinally-oriented tab-like stop members 150 are disposed along either side of the knife channel 210 of jaw member 110. Preferably, the stop members 150 may be configured in any known geometric or polynomial configuration, e.g., triangular, rectilinear, circular, ovoid, scalloped, etc., depending upon a particular purpose.

As shown in FIGS. 1B and 1C, the selectively adjustable stop member assembly 140 is located within at least one of the jaw members 110 or 120. More particularly, at least one of the jaw members, e.g., jaw member 110, includes a cavity 130 disposed therein which is dimensioned to house the stop member assembly 140. The stop member assembly 140 adjusts the distance that each stop member 150 extends from the sealing plate 112 using the controller 155, which cooperate with the stop member 150 in a plurality of ways. For example, each stop member 150 and its corresponding controller 155 may be threadably connected such that the controller 155 “unscrews” the stop member 150 to adjust the distance that the stop member 150 extends from the sealing plate 112. Other mechanical systems are also envisioned to allow selective regulation of the gap distance “G” (e.g., gearing mechanisms, camming mechanisms, pneumatic mechanisms, hydraulic mechanisms, etc.). Electromechanical systems are also contemplated (e.g., electro-mechanical actuators, ferroelectric actuators, piezo-electric actuators, piezo-ceramic actuators, magnetostrictors, thermomechanical systems [e.g., smart materials, shape memory alloys, etc.], and rotational actuators, etc.).

One version presently envisioned is a slow close activation system which is intended to include the sealing plates 112, 122, stop member(s) 150 and electrical generator 500 will now be discussed. This system involves the stop member assembly 140 being controlled automatically by the control source 300 based on the feedback received from the sensors 170a and 170b. The sensors 170a and 170b form a part of a closed-loop control system which automatically adjusts the forceps 10 prior to and/or during activation based on pre-surgical parameters and continually-sensed parameters. The sensors 170a and 170b are connected to the control source 300 (or electrosurgical generator) via cables 171a and 171b, respectively. One example of a closed-loop control system is described in commonly-owned U.S. patent application Ser. No. 10/427,832 filed on May 1, 2003 entitled “METHOD AND SYSTEM FOR CONTROLLING OUTPUT OF RF MEDICAL GENERATOR” the entire contents of which are hereby incorporated by reference herein.

In the slow-close activation system, the stop member(s) 150 are adjusted during activation based upon a continually-sensed surgical condition (e.g., tissue impedance, tissue type, tissue clarity, tissue compliance, etc.) utilizing a feed back control loop. It is envisioned that this may allow the control system to regulate the rate of closure of the sealing plates 112 and 122 upon tissue. Initially, the surgeon grasps the tissue in a customary manner and fully ratchets the forceps about the tissue within the preferred pressure ranges so that the stop member(s) 150 are extended out of the jaw members 110 (and/or 120) to achieve the desired gap distance “G”.

The preferred gap distance “G” may be selected from a look-up table during manual adjustment or determined by a computer algorithm stored within the control source 300 during automatic adjustment. For example, a relatively small gap distance “G” would be used in sealing a plurality of small blood vessels, while a larger gap distance “G” is preferable when sealing thicker tissue, such as an organ. The gap distance “G” between opposing sealing plates 112 and 122 during sealing preferably ranges from about 0.001 inches to about 0.008 inches. For smaller tissue types the gap distance is preferably between about 0.002 inches to about 0.003 inches and for larger tissue types the gap distance is preferably between about 0.004 inches to about 0.007 inches.

Once the tissue 400 is grasped between the jaw members 110 and 120 the slow closure process commences which involves retraction of the stop members 150. As the stop members 150 are retracted into the jaw members 110 and/or 120 the gap distance “G” decreases and a seal results. Therefore, the rate of closure of the sealing plates 112 and 122 is directly related to the changes in the gap distance “G”, which, in turn, depends on the rate of retraction of the stop member(s) 150 into the jaw member(s) 110 and/or 120. Hence, regulation of the retraction rate of the stop member(s) 150 directly regulates the rate of closure of the sealing plates 112 and 122.

The stop members 150 are retracted at a predetermined rate which may be adjusted manually by the surgeon (e.g., adjusting a control knob 350 shown in FIG. 2) or preferably automatically, by the control source 300 based on the feedback signals (e.g., based upon tissue thickness, tissue temperature, tissue impedance, tissue moisture, tissue clarity, tissue compliance during activation, etc.) sent by the sensors 170a and 170b. For instance, the stop members 150 can be programmed to activate in a slow close manner by automatically adjusting from a large gap distance e.g., about 0.10 inches or larger to within a preferred gap range of about 0.001 inches to about 0.008 inches during activation. As can be appreciated, this enables any surgeon to perform a slow close technique for sealing large tissue structures.

It is also envisioned that the slow close technique may be accomplished utilizing a fixed stop member configuration and spring-like sealing plates. As can be appreciated, in this instance, the stop members are configured to project or extend a fixed distance from the sealing plate or plates 112 to prevent the sealing plates from touching one another and shorting. The sealing plate, e.g., 112 (or sealing plates 112 and 122) is configured to include one or more springs 149a, 149b (or a spring assembly) which mount between the sealing plates 112 and 126 and the jaw housing 116 and 126, respectively. It is contemplated that the springs 149 allow the sealing plates 112 and 122 to slowly flex to accommodate the pressure applied to the tissue until a specified closure pressure is obtained (preferably within the above-identified working range of about 3 kg/cm2 to about 16 kg/cm2). As can be appreciated, the spring rates can be predetermined for optimal tissue effect based upon tissue type or tissue thickness. In addition, mechanical features may be included which allow the spring tension rates to be adjusted according to sensory feedback information from the generator via sensors 170a and 170b or manual input from the surgeon.

It is envisioned that any type of spring 149a, 149b may be utilized to accomplish this purpose or, alternatively, a layer of visco-elastic or elastomeric or smart material may be disposed between the sealing plates and the jaw housing to provide a specified spring rate. In this instance, gamma radiation sterilization techniques would obviously compromise the visco-elastic or elastomeric material and, as such, other sterilization techniques are envisioned that would maintain the integrity of the visco-elastic or elastomeric material, e.g., ethylene oxide sterilization.

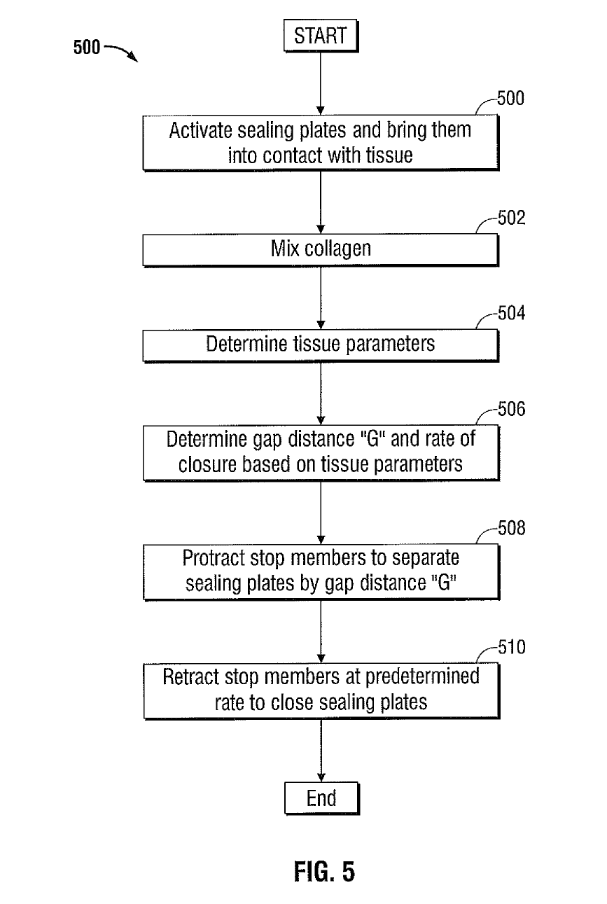

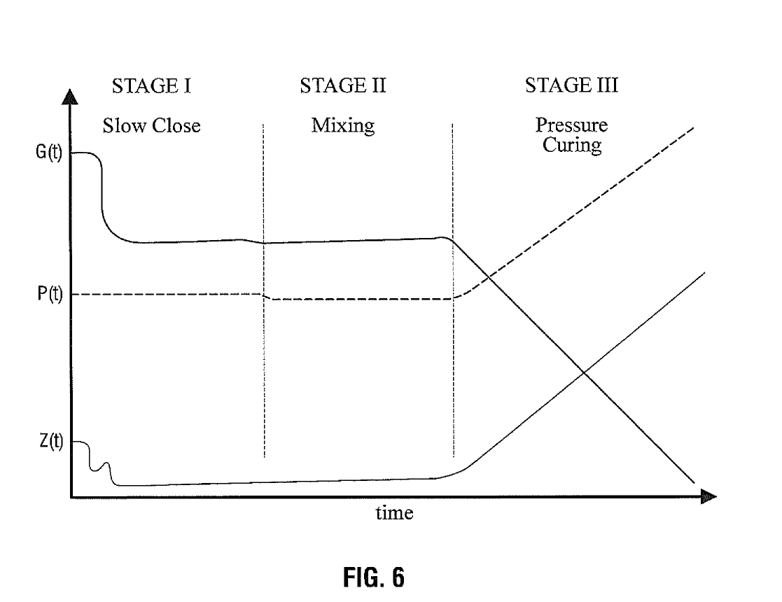

The sealing method according to the present disclosure is shown in FIG. 5. In addition, FIG. 6 shows a graph illustrating the changes that are contemplated to occur to collagen when it is subjected to sealing using the method of FIG. 5. Line G(t) represents the gap distance “G” as it changes over time, line P(t) represents the pressure applied to the tissue being sealed over time, and line Z(t) represents the electrosurgical energy applied during a specified time period.

In step 500, the forceps 10 grasps and begins to apply pressure to the tissue 400 using the jaw members 110 and 120. This is shown as Stage I in FIG. 6, during which time the sealing plates 112 and 122 are activated and are in contact with the tissue 400 but are not fully closed. This is represented by the sharp decline in the line G(t) during Stage I, which then rapidly levels off. When the sealing plates 112 and 122 contact the tissue 400 electrosurgical energy is applied thereto and the collagen contained therein is denatured and becomes more mobile (i.e., liquefies). Although electrosurgical energy is being applied, little pressure is applied to create a seal, this is shown by a straight horizontal line P(t). Simultaneously, the water contained within the tissue 400 is allowed to escape from the sealing site. As a result, the peak temperature at which a seal is created is reduced.

In step 502, the previously melted collagen is mixed in order to allow for its structural components (e.g., polymers) to intertwine as shown in Stage II. Mixing can be achieved by applying electrosurgical energy of predetermined frequency and to the sealing site through the sealing plates 112 and 122 under a predetermined pressure. The optimum frequency and amplitude of the waves depends on the collagen structures which are being mixed and may be automatically controlled as specified above. This is shown as Stage II, where the line G(t), line P(t), and line Z(t) are all generally unchanged, representing that the gap distance, the pressure, and the electrosurgical energy remain generally constant.

Once the collagen is mixed, it is further cured by applying electrosurgical energy and pressure as shown in Stage III. During Stage III the gap distance “G” decreases at a predetermined rate (e.g., the rate of closure is the slope of the line G(t), while the pressure (e.g., line P(t)) and the electrosurgical energy (e.g., line Z(t)) are increased. The pressure is preferably increased at a rate that is slow enough to result in an effective seal but not fast enough to force the forming collagen mass outside of the sealing site. As discussed above, one of the presently envisioned ways the rate at which the gap distance “G” and the sealing plates 112 and 122 are closed is controlled by the control source 300 through the stop member assembly 140, which retracts the plurality of the stop members 150 through the controllers 155. This rate at which the stop member assembly 140 decreases the distance gap “G” may be determined automatically based on the readings of the sensor assembly 170a and 170b.

In step 504, the sensors 170a and 170b sense a parameter such as tissue type, tissue thickness, tissue compliance, and/or tissue impedance and transmit that information to the control source 300. Based on the algorithms and data contained therein, in step 506, the control source 300 selects the ideal gap distance “G” for the tissue to be sealed as well as the rate at which the sealing plates 112 and 122 will close. This may also directly relate to the ideal rate of closure pressure. These calculations are transmitted to the stop member assembly 140 which, in step 508, extends or protracts the stop members 150 so that the sealing plates 112 and 122 are separated by the gap distance “G” once the jaw members 110 and 120 are closed. Once this is accomplished, in step 510, the sealing plates 112 and 122 close at the rate determined by the source controller 300, i.e., the stop member assembly 140 signals the controllers 155 to retract the stop members 150 at the predetermined rate ensuring that the rate is slow enough to retain the collagen mass at the site resulting in an effective seal.

It is envisioned that step 508 may be eliminated in the instance where the stop members 150 or stop member assembly 140 is configured to return to a preset extended condition relative to the sealing plates 112 and 122 each time the jaw members 110 and 120 are opened to grasp/manipulate tissue. It is also envisioned that the stop members may manually or automatically be extended or locked for non-slow close sealing such as those procedures described in any of the aforementioned commonly owned applications.

The apparatus and method according to the present disclosure allow for tissue sealing procedures which retain the collagen at the sealing site which is known to enhance the consistency, effectiveness, and strength of tissue seals. This is accomplished by using a slow close activation to initially denature the collagen and then close the sealing plates under pressure at a predetermined rate with limited extrusion of the cured and mixed collagen mass from the sealing site which contributes to an effective and uniform seal.

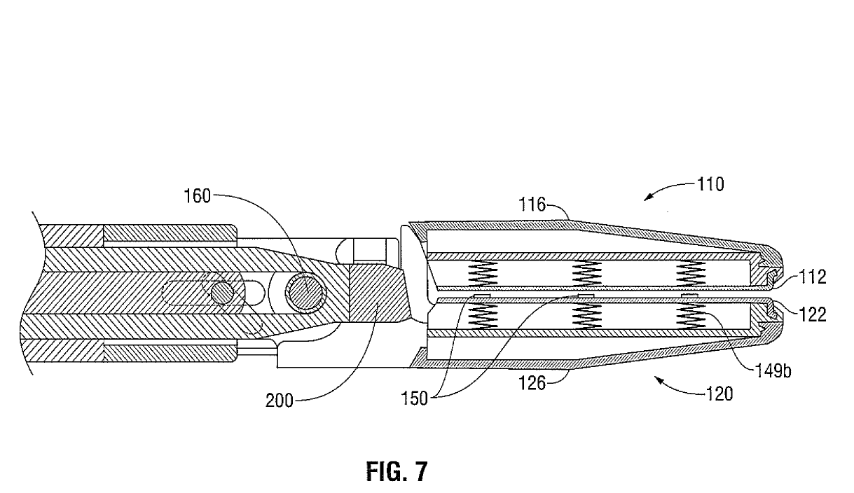

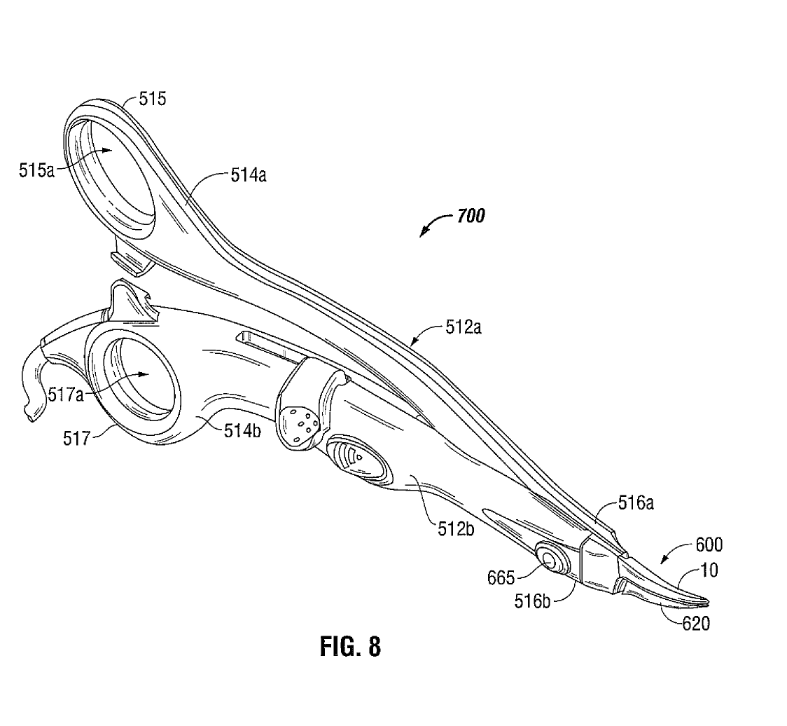

From the foregoing and with reference to the various figure drawings, those skilled in the art will appreciate that certain modifications can also be made to the present disclosure without departing from the scope of the same. For example and as mentioned above, it is contemplated that any of the slow closure techniques, methods and mechanisms disclosed herein may be employed on an open forceps such as the open forceps 700 disclosed in FIG. 8. The forceps 700 includes an end effector assembly 600 which attaches to the distal ends 516a and 516b of shafts 512a and 512b, respectively. The end effector assembly 600 includes pair of opposing jaw members 610 and 620 which are pivotally connected about a pivot pin 665 and which are movable relative to one another to grasp vessels and/or tissue. A stop member assembly such as the stop member assembly 140 described with respect to FIGS. 1-7 and/or a series of sensors 170a and 170b may be disposed within the end effector 600 to create a slow close option for the surgeon. In addition, the generator (not shown) which supplies power to the forceps 700 may be configured to automatically regulate the stop member assembly 140 (or other types of slow close mechanisms described above) or the surgeon may opt to manually control the closing of the seal plates onto the tissue as described above.

Each shaft 512a and 512b includes a handle 515 and 517, respectively, disposed at the proximal end 514a and 514b thereof which each define a finger hole 515a and 517a, respectively, therethrough for receiving a finger of the user. Finger holes 515a and 517a facilitate movement of the shafts 512a and 512b relative to one another which, in turn, pivot the jaw members 610 and 620 from an open position wherein the jaw members 610 and 620 are disposed in spaced relation relative to one another to a clamping or closed position wherein the jaw members 610 and 620 cooperate to grasp tissue or vessels therebetween. Further details relating to one particular open forceps are disclosed in commonly-owned U.S. application Ser. No. 10/962,116 filed Oct. 8, 2004 entitled “OPEN VESSEL SEALING INSTRUMENT WITH CUTTING MECHANISM AND DISTAL LOCKOUT”, the entire content of which being incorporated by reference herein.

In addition, it is also contemplated that the presently disclosed forceps may include an electrical cutting configuration to separate the tissue either prior to, during or after cutting. One such electrical configuration is disclosed in commonly-assigned U.S. patent application Ser. No. 10/932,612 entitled “Vessel Sealing Instrument With Electrical Cutting Mechanism” the entire contents of which being incorporated by reference herein.

Furthermore, it is envisioned that the forceps 10 or 700 may be configured to include a manual slow close mechanism, rotating wheel or slide which upon manual activation thereof retract the stop members relative to the sealing plates after the handle has been ratcheted and during activation. Moreover, another method may allow the surgeon to grasp and close the forceps about the tissue (within the specified pressure range) and upon activation of the switch (foot switch or hand switch) the stop members automatically retract based upon sensed surgical conditions or a preset algorithm or by preset electro-mechanical action.

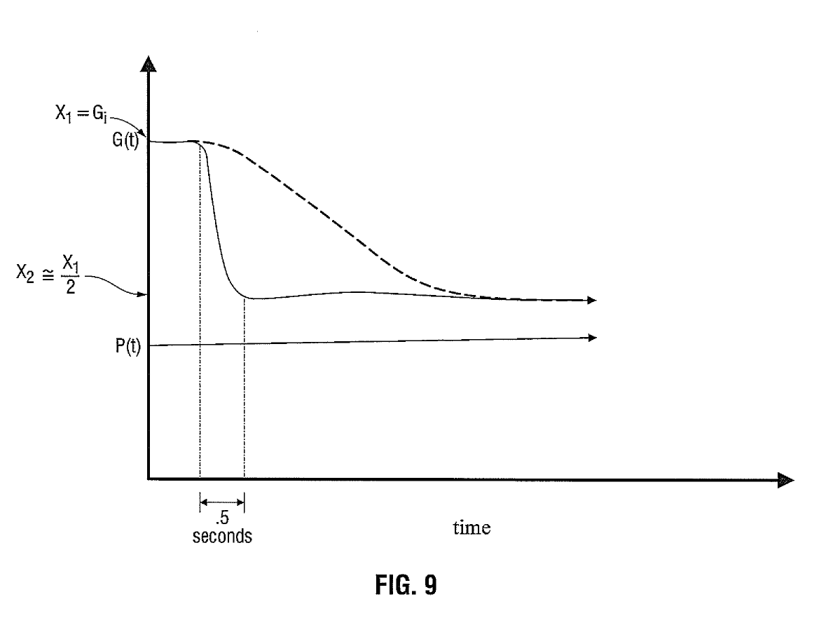

FIG. 9 shows a graph illustrating an embodiment of the changes occurring to tissue during use of the endoscopic bipolar forceps of FIGS. 1A-4 and 7-8 according to an embodiment of the present disclosure. G(t) illustrates the gap distance and is shown as a solid line or as a dotted line. P(t) illustrates the pressure applied to tissue held between electrosurgical jaws. The solid line G(t) is representative of electrosurgical energy being applied by an electrosurgical generator without using gap feedback, i.e., constant power, voltage, current or other constant electrosurgical parameter. Note that the majority of the drop in gap distance occurs within a time window of about one-half a second.

In accordance to an embodiment of the present disclosure, while constant pressure is applied to tissue with electrosurgical bipolar forceps, the electrosurgical energy communicated through tissue is adjusted such that the sensed gap distance (as sensed by a gap sensor 170 (see FIG. 1b)) decreases by about a predetermined amount as a function of time. Therefore, the electrosurgical energy is applied to control the decrease in gap distance while constant pressure is applied. This is represented by the dotted line representing G(t). The electrosurgical energy is therefore adjusted to control the “closing rate” of the gap distance “G” (see FIG. 2), and may be an arbitrary G(t). However, in some embodiments, it may be advantageous to more slowly decrease the gap distance than as indicated by the solid line G(t). For example, note that along dotted-line G(t) the sensed gap distance decreases by a constant rate of gap distance (or approximately) for a substantial amount of time.

X1 represents an initial gap distance “G” as indicated by the gap sensor 170. Electrosurgical energy is applied such that the gap distance “G” reaches about X2, which is about one-half of X1. Additionally, X2 is reached in greater than about one-half of a second. The initial gap distance, X1, may also be utilized to determine initial electrosurgical energy parameters, such as an initial frequency, an initial voltage, an initial current, an initial duty cycle, an initial power, an initial input impedance and the like. Additionally or alternatively, the initial gap distance may also be used to determine one or more of tissue type, jaw fill, tissue density, tissue compliance, tissue thickness, tissue hydration, tissue impedance, tissue impedance per unit volume and the like; test pulses or initial electrosurgical energy may be applied to the grasped tissue to facilitate these determinations.

The electrosurgical generator 500 generates the electrosurgical energy (see FIG. 3) utilizing a feedback control algorithm 215 (see FIG. 2). The feedback control algorithm 215 uses the gap distance “G” as an input and the electrosurgical energy as an output. The feedback control 215 algorithm may be a proportional-integral-derivative control algorithm (referred to herein as a P-I-D control algorithm). The control algorithm 215 can vary one or more parameters of the electrosurgical energy including frequency, voltage, current, duty cycle, power, input impedance and the like, of the electrosurgical energy to control the gap distance. Each of the jaw members 110 and 120 may have one or more adjustable stop members 150 coupled to a controller that facilitates the electrosurgical energy control of the gap distance “G” as shown by the dotted G(t) of FIG. 9.

The gap sensor 170 may be an optical gap sensor 170. For example, the optical gap sensor 170 transmits one or more optical wavelengths through tissue. In one embodiment of the optical gap sensor 170, two optical wavelengths are used with one optical wavelength being readily absorbed by water or other material commonly found in tissue. The second optical wavelength can be indifferent to the water or the other material readily absorbed by the first optical wavelength. The two wavelengths may be compared to each other to measure the gap distance. Additionally or alternatively, optical reflectance, spectral absorption, spectral reflectance and the like may be utilized by the optical gap sensor.

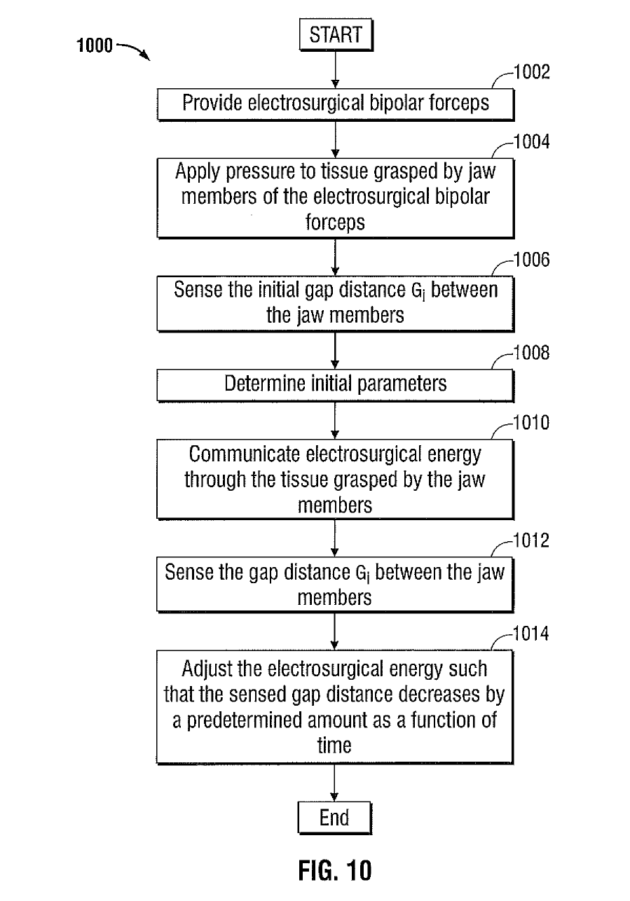

FIG. 10 shows a flow chart of a sealing method 1000 using the endoscopic bipolar forceps (e.g., the one shown in FIGS. 1A-4 and 7-8) according to the present disclosure. Method 1000 includes steps 1002 through 1014. Although steps 1002 through 1014 are shown having a sequential order, it is envisioned, in other embodiments, other orderings or sequencings may be used. For example, any one of steps 1002 through 1014 may occur simultaneously with any other step (or steps). Additionally or alternatively, any combination of serial or parallel ordering of steps 1002 through 1014 may be used.

Step 1002 provides electrosurgical bipolar forceps, e.g., electrosurgical bipolar forceps 10 of FIG. 1B. Step 1004 applies pressure to tissue grasped by jaw members 110 and 120. The pressure of step 1004 may be implemented manually or automatically, and may utilize gearing mechanisms, camming mechanisms, pneumatic mechanisms, hydraulic mechanisms and the like (not shown). Step 1006 senses the initial gap distance “Gᵢ” between the jaw members 110 and 120. The initial gap distance “Gᵢ” may be sensed by a gap sensor 170, such as an optical gap sensor 170. Step 1008 determines initial parameters such as an initial frequency, an initial voltage, an initial current, an initial duty cycle, an initial power and an initial input impedance, and the like. Step 1010 communicates electrosurgical energy through the tissue grasped by the jaw members 110 and 120. Step 1012 senses the gap distance “G” between the jaw members 110 and 120. Step 1014 adjusts the electrosurgical energy such that the sensed gap distance “G” decreases by a predetermined amount (e.g., about one-half of the initial gap distance) as a function of time, e.g., the decrease occurs after about one-half second. The electrosurgical energy may be adjusted by adjusting frequency, voltage, current, duty cycle, power, input impedance and the like.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical system for sealing tissue, comprising:
electrosurgical bipolar forceps including:
at least one shaft member having an end effector assembly disposed at a distal end thereof the end effector assembly including jaw members movable from a first position in spaced relation relative to one another to at least one subsequent position, wherein the jaw members are adapted to cooperate to grasp tissue therebetween, each of the jaw members including a sealing plate that communicates electrosurgical energy through tissue held therebetween; and
at least one gap sensor adapted to sense a gap distance between the jaw members; and
an electrosurgical generator operatively coupled to the electrosurgical bipolar forceps and configured to generate the electrosurgical energy, wherein the electrosurgical generator is in operative communication with the at least one gap sensor and is adapted to monitor a sensed gap distance therefrom, wherein the electrosurgical generator generates electrosurgical energy based on the sensed gap distance as a function of time.

2. The electrosurgical system according to claim 1, wherein the electrosurgical generator monitors the at least one gap sensor to determine an initial gap distance.

3. The electrosurgical system according to claim 2, wherein the electrosurgical generator generates electrosurgical energy to tissue such that the gap distance between the jaw members decreases by a predetermined fraction of the initial gap distance after a predetermined amount of time.

4. The electrosurgical system according to claim 3, wherein the predetermined fraction of the initial gap distance is about one-half of the initial gap distance.

5. The electrosurgical system according to claim 3, wherein the predetermined about of time is greater than about one-half of a second.

6. The electrosurgical system according to claim 2, wherein the electrosurgical generator utilizes the initial gap distance to determine at least one initial parameter of the electrosurgical energy.

7. The electrosurgical system according to claim 6, wherein the at least one initial parameter is at least one of an initial frequency, an initial voltage, an initial current, an initial duty cycle, an initial power and an initial input impedance.

8. The electrosurgical system according to claim 2, wherein the electrosurgical generator utilizes the initial gap distance to determine at least one of tissue type, jaw fill, tissue density, tissue compliance, tissue thickness, tissue hydration, tissue impedance and tissue impedance per unit volume.

9. The electrosurgical system according to claim 1, wherein the electrosurgical generator utilizes a feedback control algorithm with the gap distance being an input into the feedback control algorithm and the electrosurgical energy being an output of the feedback control algorithm.

10. The electrosurgical system according to claim 9, wherein the feedback control algorithm is a P-I-D control algorithm.

11. The electrosurgical system according to claim 1, wherein the electrosurgical generator generates the electrosurgical energy such that the sensed gap distance decreases by a predetermined amount as a function of time when the about constant pressure is applied to the grasped tissue by varying at least one parameter of the electrosurgical energy.

12. The electrosurgical system according to claim 11, wherein the at least one parameter of the electrosurgical energy is at least one of a frequency, a voltage, a current, a duty cycle, a power and an input impedance.

13. The electrosurgical system according to claim 1, wherein at least one of the sealing plates of one of the jaw members includes at least one adjustable stop member coupled to at least one controller of the electrosurgical generator, the at least one adjustable stop member configured to separate the sealing plates by a predetermined gap distance and the at least one controller configured to adjust the at least one adjustable stop member to close the sealing plates in accordance with the function of time.

14. The electrosurgical system according to claim 1, wherein the at least one gap sensor is an optical gap sensor.

15. A method for sealing tissue, comprising:
providing electrosurgical bipolar forceps comprising:
at least one shaft member having an end effector assembly disposed at a distal end thereof, the end effector assembly including jaw members movable from a first position in spaced relation relative to one another to at least one subsequent position, wherein the jaw members are adapted to cooperate to grasp tissue therebetween, each of the jaw members including a sealing plate adapted to contact the grasped tissue; and
at least one gap sensor adapted to sense a gap distance between the jaw members; and
applying pressure to the tissue grasped by the jaw members,
sensing the gap distance between the jaw members;
communicating electrosurgical energy through the tissue grasped by the jaw members; and
adjusting the electrosurgical energy such that the sensed gap distance decreases by a predetermined amount as a function of time.

16. The method according to claim 15, wherein the predetermined amount is about one-half of an initial gap distance.

17. The method according to claim 15, wherein the adjusting step adjusts the electrosurgical energy such that the sensed gap distances decreases by the predetermined amount after about one-half of a second.

18. The method according to claim 15, further comprising:
sensing an initial gap distance between the jaw members.

19. The method according to claim 18, further comprising:
determining at least one of an initial frequency, an initial voltage, an initial current, an initial duty cycle, an initial power and an initial input impedance utilizing the initial gap distance between the jaw members.
